(19) United States
(12) Patent Application Publication
(10) Pub. No.: US 2019/0183647 A1
(43) Pub. Date: Jun. 20, 2019

(54) Non-Planar Cardio Vascular Support Prosthesis

(71) Applicant: The Trustees of the University of Pennsylvania, Philadelphia, PA (US)

(72) Inventors: Ivan S. Salgo, Andover, MD (US); Joseph H. Gorman, III, Lower Gwynedd, PA (US); Robert C. Gorman, Lower Gwynedd, PA (US)

(21) Appl. No.: 16/106,795

(22) Filed: Aug. 21, 2018

Related U.S. Application Data
(63) Continuation of application No. 10/283,761, filed on Oct. 30, 2002, now abandoned.
(60) Provisional application No. 60/334,780, filed on Nov. 1, 2001.

(51) Int. Cl. A61F 2/24

(52) U.S. Cl. CPC A61F 2/2448; A61F 2/2445

(57) ABSTRACT

An annuloplasty ring comprising an elongated curved member defining a ring-shape having an at rest size and shape to fit against the annulus of the mitral valve in a heart, the member having a substantial saddle shape wherein the annular height to width ratio is in the range of 5% to 50%.

### BACKGROUND OF THE INVENTION

The mitral valve is a complex structure whose competence relies on the precise interaction of annulus, leaflets, chordae, papillary muscles and left ventricle (LV). Pathologic changes in any of these structures can lead to valvular insufficiency. Myxomatous leaflet/chordal degeneration, and dilated ischemic cardiomyopathy secondary to chronic post infarction ventricular remodeling are among most common mechanisms producing mitral regurgitation (MR). These two disease processes account for about 78% of all cases of MR treated surgically.

Ring annuloplasty was recognized as an essential component of mitral valve repair at least as early as 1969. The goal of an annuloplasty ring (sometimes referred to simply as “ring”) is to remodel the annulus back to a more normal geometry, decreasing tension on suture lines, increasing leaflet coaptation and preventing progressive annular dilatation. Surgeons with experience in mitral valve repair agree that restoration of normal annular shape with a ring is an essential component to most if not all mitral valve repairs. In fact it has been shown that repairs without a ring are less durable.

FIG. 1 is an elevation view of a known annuloplasty ring 100, typically referred to as a Carpentier Ring. The device shown in FIG. 1 is more fully described in Carpentier U.S. Pat. No. 3,656,185, incorporated herein by reference. In elevation the prosthesis 100 closely follows the shape of the base of the valve with a circular or oval shape with a flattened portion at 102. It is noteworthy that Carpentier Rings are substantially planar. The flattened portion is traditionally one quarter to one half the length of the ring 100. The flattened portion 102 corresponds to the curvature of the large cusp and the complementary zone corresponds to the curvature of the small cusp. Such rings have an axis of symmetry. Along the axis of symmetry, the ring has a width of between 15 mm and 30 mm. Perpendicular to the axis of symmetry, the ring has a length of between 20 mm and 40 mm.

The last 30 years has seen the development of a wide range of annuloplasty devices and techniques. Recent variations in ring design have focused on flexibility, for example, Carpentier et al U.S. Pat. No. 4,055,861. Carpentier et al U.S. Pat. No. 5,061,277 describes a ring having stiff segments interspersed with flexible segments.

The impetus for flexible rings is that the normal systolic contraction of the annular orifice is important to valvular and left ventricular function. However, it has been difficult to design a ring that is flexible enough to allow annular contraction and still meet all of the geometrical restorative ring requirements. Although the durability of these repairs using these devices has been acceptable, there are problems.

Most significantly, there is a significant long-term failure rate. Animal studies have shown that flexible rings allow very little systolic annular contraction. Careful analysis of recent long term follow up of mitral repairs shows that the flexible rings may have a slightly higher failure rate than non-flexible rings. The present inventors believe that this indicates that firm annular support and restoration of normal geometry are more import contributors to repair durability than maintaining annular flexibility.

To date, known mitral valular support prosthesis comprise largely planar rings. In other words, looking at a side view, known rings lie flat as with the various Carpentier rings. Medical professionals have known that the mitral valve, as it exists in nature, is not planar, rather it is saddle shaped. See Levine R A, Handschumacher M D, Sanfilippo A J et al. Three-Dimensional Echocardiographic Reconstruction of the Mitral Valve, with Implications for the Diagnosis of Mitral Valve Prolapse, circulation 1989; 80:589-598. Regardless of this knowledge, makers of mitral valular support prosthesis still predominately provide rings having a largely planar profile. At least one known prosthesis, the Carpentier-Edwards Physio Annuloplasty Ring currently marketed by EDWARDS LIFESCIENCES, has a non-planar profile. This ring has a mild saddle shape with downwardly extending lobes. While this ring is slightly closer to the true shape of the mitral valve than the predominate planar configurations, it is still not the shape of the mitral valve as it exists in mature.

The present inventors have identified a set of criteria that can be used in the design and fabrication of cardiac valular support prosthesis that closely mimic the shape of the natural mitral valve.

### BRIEF DESCRIPTION OF THE DRAWINGS

An understanding of the present invention can be gained from the following detailed description of the invention, taken in conjunction with the accompanying drawings of which:

FIG. 1 is an elevation of a known annuloplasty ring, typically referred to as a Carpentier Ring.

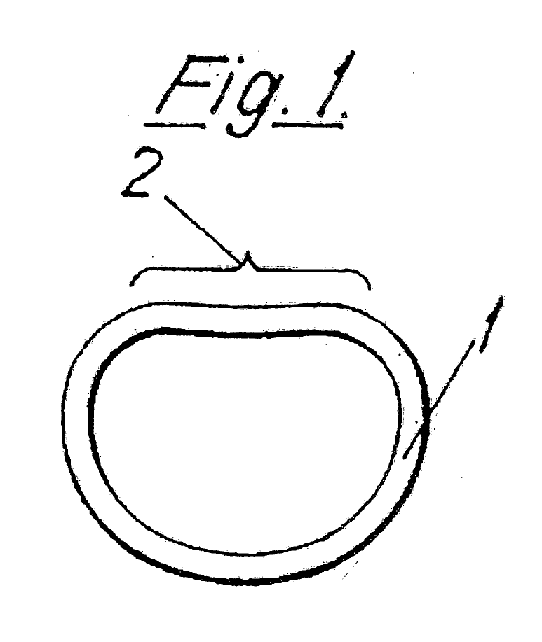

FIGS. 2a through 2c are charts useful for explaining equations used to design rings in accordance with a preferred embodiment of the present invention.

FIG. 3 is an isometric view of an annuloplasty ring shaped in accordance with the preferred embodiment of the present invention.

FIG. 4 is a plan view of the annuloplasty ring shown in FIG. 3.

FIG. 5 is a side view of the annuloplasty ring shaped shown in FIG. 3.

FIG. 6 is a side view of the annuloplasty ring s shown in FIG. 3.

FIG. 7 is an isometric view of an annuloplasty ring shaped in accordance with the preferred embodiment of the present invention.

FIG. 8 is an isometric view of an annuloplasty ring shaped in accordance with the preferred embodiment of the present invention.

### DETAILED DESCRIPTION

Reference will now be made in detail to the present invention, examples of which are illustrated in the accompanying drawings, wherein like reference numerals refer to like elements throughout.

Studies have shown that complex mitral valve suffer durability problems. That is, repairs requiring chordal transfer /shortening and leaflet suture lines. In many cases these long-term failures are the result of disruption at leaflet, chordal or annular suture lines. Frank chordal rupture also leads to recurrent mitral regurgitation. Such failure mechanisms suggest stress and the resulting strain as an etiologic factor.

Leaflet, chordal and annular stress are directly proportional to load (the difference between systolic LV and left atrial pressure) and leaflet area and inversely proportional to leaflet curvature (Laplace's law). Mitral leaflet curvature is in turn governed by two mechanisms. The most obvious and well described is leaflet billowing. The leaflet area of the human mitral valve is more than twice its annular area. This excess tissue allows the leaflets to billow (curve concave towards the LV) under a systolic load and still maintain adequate coaptation. This billowing induced curvature acts to reduce stress and strain on the leaflets, annulus and chordae.

Unfortunately, most currently available annuloplasty devices are essentially flat. When implanted the rigid and semi-rigid devices only restore the annular geometry in two dimensions. The height of the annulus is totally obliterated, thereby placing increased stress and subsequent strain on the repair by the diminished leaflet curvature imposed by annular flattening. The stress induced by annular flattening is likely exacerbated in repairs requiring leaflet resection, which further decreases leaflet curvature by diminishing tissue available for billowing.

The present Inventors have discovered that the non-planer shape of the mitral annulus may reduce leaflet stress by imparting a second but equally important form of leaflet curvature. The greatest reduction in leaflet stress is believed to occur when the annular height to width ratio (AHCWR—a measure of non planarity) is 15 to 20%. As noted above, it has been reported that humans (and several other mammalian species) have mitral annuli that are naturally saddle shaped with AHCWR values in this 15 to 20% range. However, an AHCWR in the range of 5% to 50% would be within the scope of the present invention. The inventors have discovered that such a shape is dynamically accentuated during systolic loading and is significantly reduced in ovine models of ischemic mitral regurgitation.

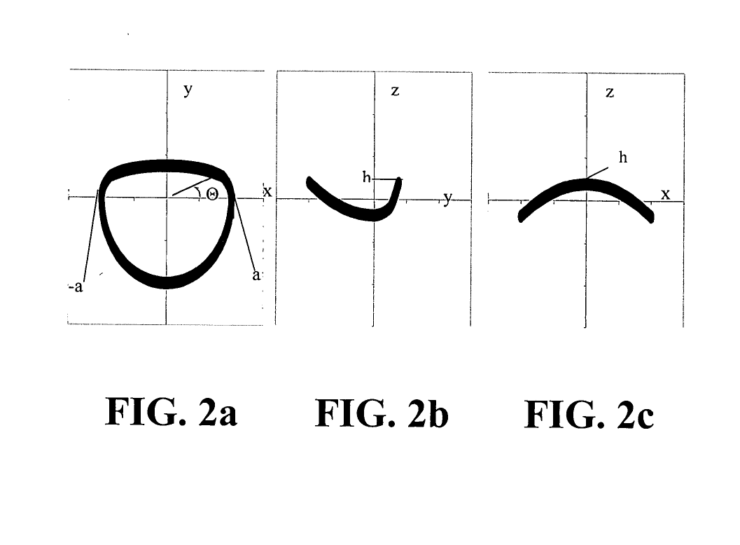

FIGS. 2a through 2c are charts useful for explaining equations used to design rings in accordance with a preferred embodiment of the present invention. The present Inventors have discovered a series of equations that can be used to form annuloplasty rings with shapes that mimic the natural shape of the mitral valve that, as discussed above, is believed to increase durability of valve repairs. Using equations 1 though 3, a suitable prosthesis (e.g. ring) can be described, thereby facilitating fabrication thereof. FIG. 2 shows the relationship between the coefficients “a” and “h” and “Θ.” “e₁” and “e₂” are not shown in FIG. 2 but are functions of “Θ” and will be further described hereinafter.

X=a cos(Θ) Equation 1:

Y=a e₁(e₂+sin(Θ)) Equation 2:

Z=−a h cos(2Θ) Equation 3:

Depending on the specification of the coefficients a, e₁, e₂ and h Equations 1-3 can describe a wide range of “ring” designs—from a simple circle of radius 1 (a=e₁=1, e₂=0, and h=0) to a somewhat complicate hyperbolic parabloid with varying eccentricity and size.

For every Θ from 0 to 360° the X, Y, Z coordinates for the ring can be defined for any given set of coefficients (a, e₁, e₂, and h). Once these coordinates have been generated any and all size specifications for a particular ring can be calculated

“a” is a scaling or sizing term. The value of “a” sets the size of the ring. In practice “a” is be proportionate to a patients so called intercommisural distance (this is how current rings are sized). Generally, 2a=the intercommisural distance.

“h” defines the “height” or non-planarity of the ring (the Z dimension) and corresponds to the annular height to commissural with ratio (AHCWR). Based on the discoveries of the present inventors, the preferred range for “h” is between 15 and 20 percent. As noted above, “h” may vary from 5% to 50%.

The coefficients e₁ and e₂ specify the eccentricity of the ellipse in the XY plane and are functions of Θ. In accordance with perhaps the preferred embodiment, they vary with Θ as follows:

TABLE 1

| | e1 | e2 |
|---|---|---|
| 0 ≤ Θ < 30 | 0.5 | 0.0 |
| 30 ≤ Θ < 150 | 0.25 | 0.5 |
| 150 ≤ Θ < 180 | 0.5 | 0.0 |
| 180 ≤ Θ < 360 | 1.0 | 0.0 |

The values set forth in TABLE 1 produce a D-shaped ring similar (at least as seen in from a plan view) to the classic Carpentier ring. Those of ordinary skill in the art will recognize that the values for e₁ and e₂ may be varied to produce a variety of shapes, as seen in a plan view. Thus, while a D-shaped ring will been shown and described as the preferred embodiment, other shapes may be utilized. More to the point the particulars of the shape of the ring in the plan view is largely outside the scope of the present invention. However, those of ordinary skill in the art will be able to produce most desired shapes using equations 1 through 3. For example, a circular ring is produced using the following table:

TABLE 2

| | e1 | e2 |
|---|---|---|
| 0 ≤ Θ < 30 | 1.0 | 0.0 |
| 30 ≤ Θ < 150 | 1.0 | 0.0 |
| 150 ≤ Θ < 180 | 1.0 | 0.0 |
| 180 ≤ Θ < 360 | 1.0 | 0.0 |

Similarly an elliptical ring can be produced using the following table:

TABLE 3

| | e1 | e2 |
|---|---|---|
| 0 ≤ Θ < 30 | 0.5 | 0 |
| 30 ≤ Θ < 150 | 0.5 | 0 |
| 150 ≤ Θ < 180 | 0.5 | 0 |
| 180 ≤ Θ < 360 | 0.5 | 0 |

The remainder of the discussion will focus on a D-shape ring.

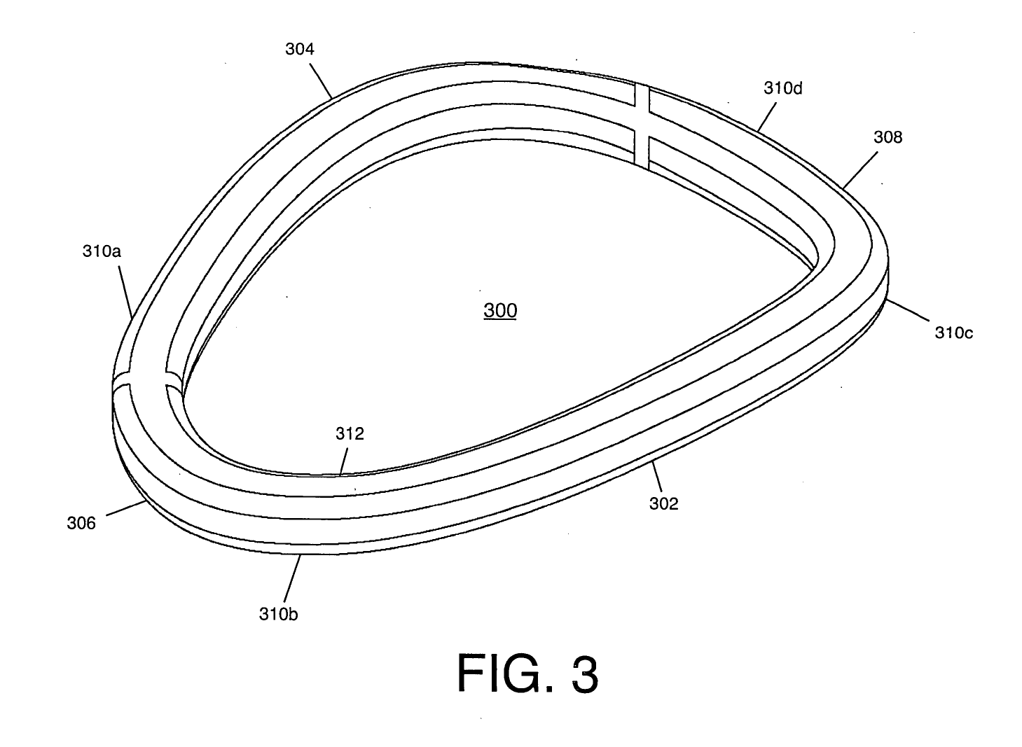

FIG. 3 is an isometric view of an annuloplasty ring 300 shaped in accordance with the preferred embodiment of the present invention. Certain artistic license has been taken with FIG. 3 to emphasis the 3-D shape of the present invention. The lines used to give the illusion of depth are, of course, not present in rings constructed in accordance with the present invention. More to the point, the ring 300 comprises an elongated curved member defining a ring-shape having an at-rest size and shape to recreate the annulus of cusps of a natural human heart in length, width and elevation during systole. As noted such a shape is sometimes referred to as a saddle shape. The ring 300 is generated using equations 1 through 3 along with TABLE 1 wherein h=20%.

The saddle shape of the ring 300 can be described approximately in mathematical terms as a hyperbolic parabloid. The ring has ‘negative curvature’; meaning it imparts curvature on the leaflets in two directions. Along segments 302 and 304 the ring is convex towards the ventricle and at segments 306 and 308 the ring is concave, thereby producing a′ great deal of curvature in a limited space. The extent of the curvature is controlled by the “h” parameter in Equations 1-3. At transition points 310a-310d where the ring changes from convex to concave with respect to the ventricle there is minimal annular curvature. The transition points 310a-310d correspond to points on the posterior annulus where chordal rupture is most commonly seen clinically (mid posterior leaflet—the so called P2 segment). It is the presence of such ruptures that lead the present inventors to recognize that natural annular shape is important in reducing leaflet stress and resulting strain.

Preferably, the ring 300 is formed with means for suturing the ring 300 into place. For example the ring 300 is provided with an outer flexible sheath 312, typically formed of fabric such as knitted Dacron, that can be sewn into the existing valve structure. The interior of the ring 300 can be formed of a variety of know materials. The material used to form the ring 300 and sheath 312 is beyond the scope of the present application, but will be understood by those of ordinary skill in the art.

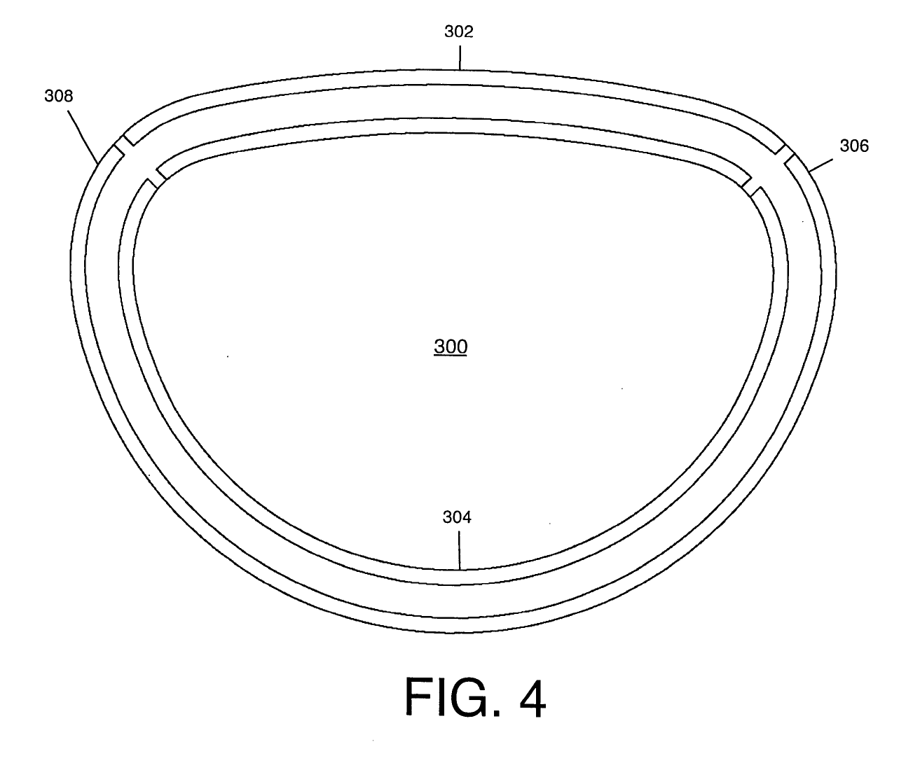

FIG. 4 is a plan view of the annuloplasty ring shown in FIG. 3. The overall D-shape of the ring 300 can be seen in this view. To be specific, it becomes apparent that segment 302 is approximately flat (e.g. reduced curvature compared with the rest of the elongated curved member), at least in the plan view. Whereas segments 304, 306 and 308 form the curved portion of the D-shape in the plan view. The extent of the D-shape can be controlled by e₁ and e₂. With the proper selection of values, the design specification can range from circular to elliptic to D-shape. The shape shown in FIG. 4 mimics the actual shape of the mitral valve.

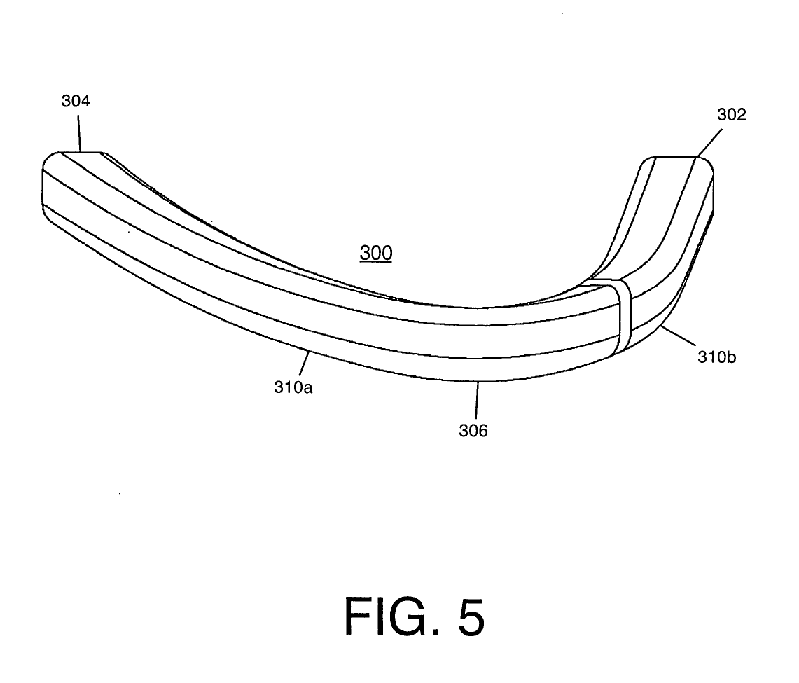

FIG. 5 is a side view of the annuloplasty ring shown in FIG. 3. More specifically, FIG. 5 shows the ring 300 as viewed from the mitral commisure to commisure. The view shown in FIG. 5 highlights the non-symmetrical curve, in the height direction, displayed by segments 306 and 308 (not seen). More specifically, the segments 306 and 308 display a greater absolute slope closer to the straight segment 302.

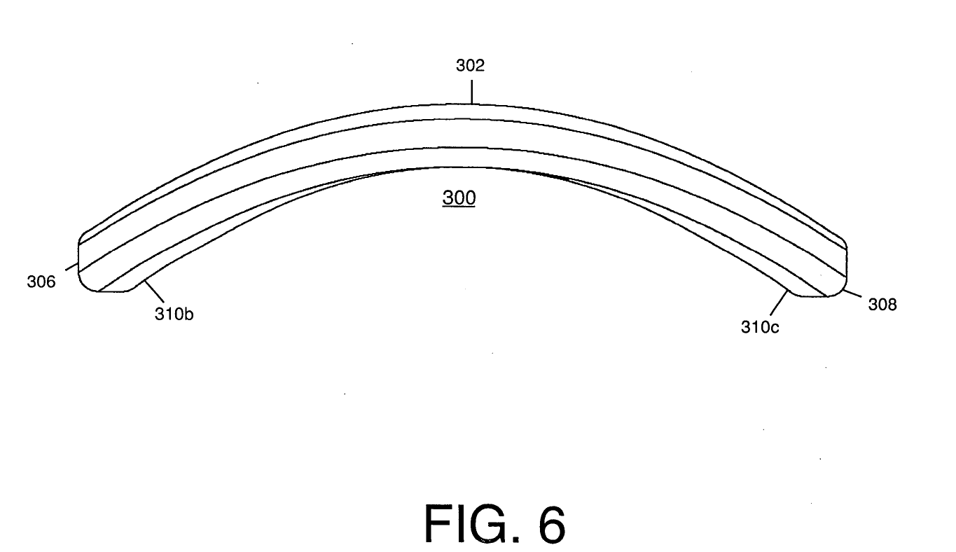

FIG. 6 is a side view of the annuloplasty ring shown in FIG. 3. More specifically, FIG. 3 shows the ring 300 as viewed from the posterior to the anterior annulus. The view shown in FIG. 6 highlights the more symmetrical curve, in the height direction, displayed by segments 302 and 304 (not seen).

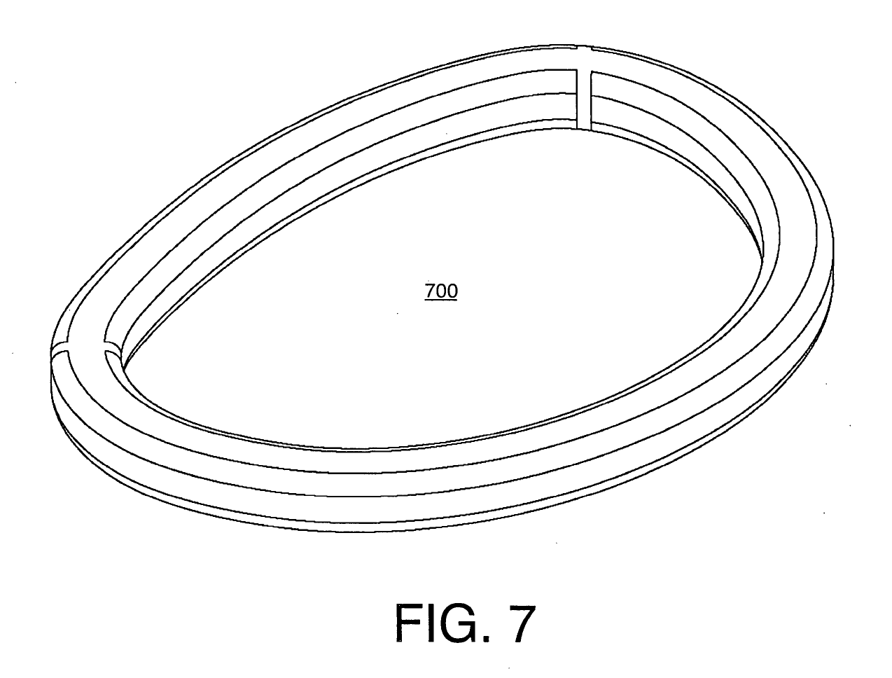

FIG. 7 is an isometric view of an annuloplasty ring 700 shaped in accordance with the preferred embodiment of the present invention. The ring 700 is produced by setting “h” to 5%.

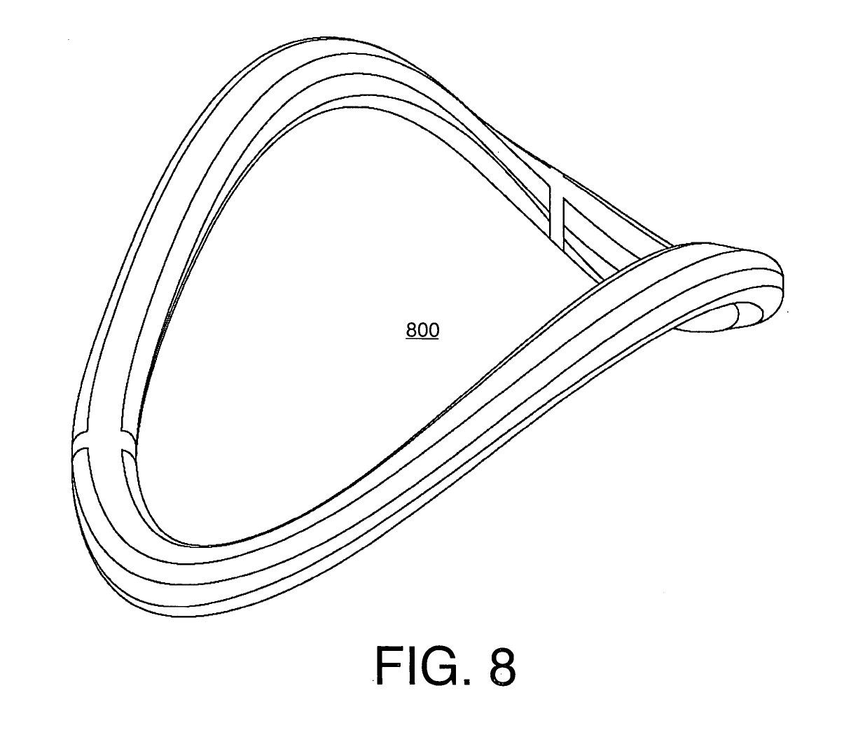

FIG. 8 is an isometric view of an annuloplasty ring 800 shaped in accordance with the preferred embodiment of the present invention. The ring 800 is produced by setting “h” to 50.

While the optimal saddle shape annuloplasty ring design would be described by equations 1, 2 and 3 with the design parameters (e1, e2, e3, h) set to the values described in Table 1, a range of shapes around this optimal design may also be beneficial. As a result they have developed a second set of Equations (4,5,and 6) which define a range of design specifications with varying combinations of the design parameters e1, e2, e3 and h, which describe other beneficial designs. As such, any annuloplasty ring with a value of h between 0.05 and 0.5 and encompassed by the range of specifications defined by Equations 4,5, and 6 is within the scope of the present invention. Equations 4, 5, and 6 describe a tube shaped member useful for generating the design of any given ring. Similar to Equations 1, 2, and 3 the Equations 4, 5, and 6 provide x, y, and z coordinates. However, unlike Equations 1-3, which define a centerline of a ring, Equations 4, 5, and 6 define an acceptable space for the ring.

$$\begin{matrix}X = ( Cos [ Θ ] ( \sqrt{2} r Cos [ Φ ] ( {e}_{1}^{2} + 16 {h}^{2} {Sin [ Θ ]}^{4} ) + \sqrt{\begin{matrix}{e}_{1}^{2} {Cos [ Θ ]}^{2} + {Sin [ Θ ]}^{2} + \\ 4 {h}^{2} {Sin [ 2 Θ ]}^{2}\end{matrix}} \cdot ( a \sqrt{\begin{matrix}2 {e}_{1}^{2} + 10 {h}^{2} + 10 {e}_{1}^{2} {h}^{2} + 15 ( - 1 + {e}_{1}^{2} ) \\ {h}^{2} Cos [ 2 Θ ] + 6 ( 1 + {e}_{1}^{2} ) {h}^{2} Cos [ 4 Θ ] - \\ {h}^{2} Cos [ 6 Θ ] + {e}_{1}^{2} {h}^{2} Cos [ 6 Θ ]\end{matrix}} + 4 \sqrt{2} {e}_{1} hr {Cos [ Θ ]}^{2} Sin [ Φ ] ) ) ) \div ( \sqrt{\begin{matrix}2 {e}_{1}^{2} + 10 {h}^{2} + 10 {e}_{1}^{2} {h}^{2} + 15 ( - 1 + {e}_{1}^{2} ) \\ {h}^{2} Cos [ 2 Θ ] + 6 ( 1 + {e}_{1}^{2} ) {h}^{2} Cos [ 4 Θ ] - \\ {h}^{2} Cos [ 6 Θ ] + {e}_{1}^{2} {h}^{2} Cos [ 6 Θ ]\end{matrix}} \cdot \sqrt{\begin{matrix}{e}_{1}^{2} {Cos [ Θ ]}^{2} + {Sin [ Θ ]}^{2} + \\ 4 {h}^{2} {Sin [ 2 Θ ]}^{2}\end{matrix}} ) & Equation 4 \\ Y = ( \sqrt{2} {e}_{1} r ( 1 + 16 {h}^{2} {Cos [ Θ ]}^{4} ) Cos [ Φ ] Sin [ Θ ] + \sqrt{\begin{matrix}{e}_{1}^{2} {Cos [ Θ ]}^{2} + {Sin [ Θ ]}^{2} + \\ 4 {h}^{2} {Sin [ 2 Θ ]}^{2}\end{matrix}} \cdot ( {ae}_{1} {e}_{2} \sqrt{\begin{matrix}2 {e}_{1}^{2} + 10 {h}^{2} + 10 {e}_{1}^{2} {h}^{2} + 15 ( - 1 + {e}_{1}^{2} ) \\ {h}^{2} Cos [ 2 Θ ] + 6 ( 1 + {e}_{1}^{2} ) {h}^{2} [ 4 Θ ] - \\ {h}^{2} Cos [ 6 Θ ] + {e}_{1}^{2} {h}^{2} Cos [ 6 Θ ]\end{matrix}} + a Sin [ Θ ] {e}_{1} \sqrt{\begin{matrix}2 {e}_{1}^{2} + 10 {h}^{2} + 10 {e}_{1}^{2} {h}^{2} + 15 ( - 1 + {e}_{1}^{2} ) \\ {h}^{2} Cos [ 2 Θ ] + 6 ( 1 + {e}_{1}^{2} ) {h}^{2} Cos [ 4 Θ ] - \\ {h}^{2} Cos [ 6 Θ ] + {e}_{1}^{2} {h}^{2} Cos [ 6 Θ ]\end{matrix}} - 4 \sqrt{2} hr {Sin [ Θ ]}^{3} Sin [ Φ ] ) ) \div ( \sqrt{\begin{matrix}2 {e}_{1}^{2} + 10 {h}^{2} + 10 {e}_{1}^{2} {h}^{2} + 15 ( - 1 + {e}_{1}^{2} ) \\ {h}^{2} Cos [ 2 Θ ] + 6 ( 1 + {e}_{1}^{2} ) {h}^{2} Cos [ 4 Θ ] - \\ {h}^{2} Cos [ 6 Θ ] + {e}_{1}^{2} {h}^{2} Cos [ 6 Θ ]\end{matrix}} \cdot \sqrt{{e}_{1}^{2} {Cos [ Θ ]}^{2} + {Sin [ Θ ]}^{2} + 4 {h}^{2} {Sin [ 2 Θ ]}^{2}} ) & Equation 5 \\ Z = ( - 4 \sqrt{2} {e}_{1}^{2} hr {Cos [ Θ ]}^{4} Cos [ Φ ] + 4 \sqrt{2} hr Cos [ Φ ] {Sin [ Θ ]}^{4} + \sqrt{\begin{matrix}{e}_{1}^{2} {Cos [ Θ ]}^{2} + {Sin [ Φ ]}^{2} + \\ 4 {h}^{2} {Sin [ 2 Θ ]}^{2}\end{matrix}} \cdot ( - ah Cos [ 2 Θ ] \cdot \sqrt{\begin{matrix}2 {e}_{1}^{2} + 10 {h}^{2} + 10 {e}_{1}^{2} + 15 ( - 1 + {e}_{1}^{2} ) \\ {h}^{2} Cos [ 2 Θ ] + 6 ( 1 + {e}_{1}^{2} ) {h}^{2} Cos [ 4 Θ ] - \\ {h}^{2} Cos [ 6 Θ ] + {e}_{1}^{2} {h}^{2} Cos [ 6 Θ ]\end{matrix}} + \sqrt{2} {e}_{1} r Sin [ Φ ] ) ) \div ( \sqrt{\begin{matrix}2 {e}_{1}^{2} + 10 {h}^{2} + 10 {e}_{1}^{2} {h}^{2} + 15 ( - 1 + {e}_{1}^{2} ) \\ {h}^{2} Cos [ 2 Φ ] + 6 ( 1 + {e}_{1}^{2} ) {h}^{2} Cos [ 4 Θ ] - \\ {h}^{2} Cos [ 6 Θ ] + {e}_{1}^{2} {h}^{2} Cos [ 6 Θ ]\end{matrix}} \cdot \sqrt{\begin{matrix}{e}_{1}^{2} {Cos [ Θ ]}^{2} + {Sin [ Θ ]}^{2} + \\ 4 {h}^{2} {Sin [ 2 Θ ]}^{2}\end{matrix}} ) & Equation 6\end{matrix}$$

Where “r” is the tube's radius (of the cloud); and “Φ” is a parametric variable that goes around the tube (a clock face perpendicular to the base saddle wire frame). “a”, “h”, e₁ and e₂ vary as set forth with Equations 1 through 3 above.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed:

1. An annuloplasty ring, comprising:
an elongated curved non-planar member having a ring shape defined in coordinates X, Y, and Z by:
X=a cos(Θ);
Y=a e₁(e₂+sin(Θ)); and
Z=−a h cos(2Θ);
wherein “a” represents a predetermined scale of the ring, “h” defines a predetermined non-planarity of the member and corresponds to an annular height divided by an annular width of the member, e₁ and e₂ specify a predetermined eccentricity of the ring in the X-Y plane, “Θ” indicates an angular displacement of each position around the member with respect to an origin of a coordinate axis on the X-Y plane,
wherein “h” has a value in the range of 0.05 to 0.5, and
wherein a, e₁, e₂ and h have values selected to mimic an actual shape of a mitral valve.

2. An annuloplasty ring as set forth in claim 1, wherein the elongated curved member has a size and shape to fit against the annulus of the mitral valve in a heart during systole.

3. An annuloplasty ring as set forth in claim 1, wherein the elongated curved member further comprises:
a frame;
compressible material surrounding said frame; and
a sheath formed about the compressible material.

4. An annuloplasty ring as set forth in claim 1, wherein “h” has a value of not less than 0.15 and not greater than 0.50.

5. An annuloplasty ring as set forth in claim 1, wherein e₁ and e₂ vary based on Θ.

6. An annuloplasty ring as set forth in claim 5, wherein e₁ and e₂ are set based on:

| | e1 | e2 |
|---|---|---|
| 0 ≤ Θ < 30 | 0.5 | 0.0 |
| 30 ≤ Θ < 150 | 0.25 | 0.5 |
| 150 ≤ Θ < 180 | 0.5 | 0.0 |
| 180 ≤ Θ < 360 | 1.0 | 0.0 |

7. An annuloplasty ring set forth in claim 6, wherein “h” has a value of not less than 0.15 and not greater than 0.50.

8. An annuloplasty ring, as set forth in claim 5, wherein the values of e₁ and e₂ are selected to form a D-shaped ring in a plan view of the annuloplasty ring.

9. An annuloplasty ring as set forth in claim 1, further comprising:
suturing means for suturing the member in place.

10. An annuloplasty ring as set forth in claim 1, wherein said member is flexible.

11. An annuloplasty ring as set forth in claim 1, wherein said member is stiff.

12. An annuloplasty ring as set forth in claim 1, wherein a portion of said member is flexible.

13. An annuloplasty ring as set forth in claim 1, wherein a portion of said member is stiff.
